(19) United States
(12) Patent Application Publication
(10) Pub. No.: US 2021/0247407 A1
(43) Pub. Date: Aug. 12, 2021

(54) NOVEL BIOMARKERS FOR RECURRENT TONSILLITIS

(71) Applicant: Westfaelische Wilhelms-Universitaet Muenster, Muenster (DE)

(72) Inventors: Christoph Spiekermann, Muenster (DE); Thomas Vogl, Muenster (DE); Johannes Roth, Muenster (DE)

(21) Appl. No.: 17/251,443

(22) PCT Filed: Jun. 6, 2019
(86) PCT No.: PCT/EP2019/064825

§ 371 Date: Dec. 11, 2020

(30) Foreign Application Priority Data
Jun. 13, 2018 (LU) LU100835

(51) Int. Cl. G01N 33/68

(52) U.S. Cl. CPC G01N 33/6893; G01N 2800/54; G01N 33/6869

(57) ABSTRACT

The present invention relates to a method of stratifying a subject having no symptoms of acute tonsillitis for tonsillectomy or tonsillotomy, to a method of diagnosing recurrent tonsillitis in a subject having no symptoms of acute tonsillitis and to a method of predicting the risk or the predisposition for recurrence of acute tonsillitis (RAT) in a subject having no symptoms of acute tonsillitis. These methods ae based on the determination of the biomarkers IL-1 β, IL-18 and S100A8/S100A9 in said subject.

### FIELD OF THE INVENTION

The present invention relates to a method of stratifying a subject having no symptoms of acute tonsillitis for the necessity of tonsillectomy or tonsillotomy, comprising (i) determining the amount of the biomarkers IL-1β, IL-18 and S100A8/S100A9 in a sample obtained from said subject, and (ii) stratifying said subject for the necessity of tonsillectomy or tonsillotomy by comparing the amount of said biomarkers with a threshold value for each biomarker. Further, the present invention provides a method of diagnosing recurrent tonsillitis in a subject having no symptoms of acute tonsillitis for the necessity of tonsillectomy or tonsillotomy, comprising (i) determining the amount of the biomarkers IL-1β, IL-18 and S100A/S100A9 in a sample obtained from said subject, and (ii) stratifying said subject for the necessity of tonsillectomy or tonsillotomy by comparing the amount of said biomarkers with a threshold value for each biomarker. The present invention also relates to a method of predicting the risk or the predisposition for recurrence of acute tonsillitis (RAT) in a subject having no symptoms of acute tonsillitis for the necessity of tonsillectomy or tonsillotomy, comprising (i) determining the amount of the biomarkers IL-1β, IL-18 and S100A8/S100A9 in a sample obtained from said subject, and (ii) stratifying said subject for the necessity of tonsillectomy or tonsillotomy by comparing the amount of said biomarkers with a threshold value for each biomarker.

### BACKGROUND OF THE INVENTION

As a part of the Waldeyer's ring, the palatine tonsils represent secondary lymphoid organs and belong to the mucosa-associated lymphoid tissue (MALT) (Hellings et al. 2000). Although they show some similarities to the spleen, lymph nodes or Peyer's patches of the gut, some unique characteristics exist for the palatine tonsil such as a partial capsule, the existence of lymphoreticular structures but an absence of afferent lymphatics (Perry et al. 1998). The palatine tonsils represent the first line in the defense of oropharyngeal pathogens and several reports have described their immunological significance (Sudan et al. 1987, Brandtzaeg et al. 1992, Paulussen et al. 2000; Nave et al. 2001).

However, in some cases the palatine tonsils provoke increased inflammatory reactions associated with symptoms of a sore throat and a general malaise. Acute tonsillitis is the most common reason for emergency admission to otolaryngology service and recurrence of acute tonsillitis (RAT) and of sore throat episodes in general generate a substantial utilization of medical resources (Bird et al. 2014, Mui et al. 1998). The reason for the recurrence of acute tonsillitis in some patients still remains unclear. Tonsillectomy and tonsillotomy are very common procedures and several reports have described the benefits concerning recurrent tonsillitis and recurrent sore throat episodes (Mui et al. 1998, Morad et al. 2017, Burton 2014, Senska 2015).

Inflammatory processes are characterized and regulated by release of various cytokines and chemokines. The importance and influence of major molecules such as TNF-α, IL-1ß and IL-6 on inflammatory processes is well-reported but analyzing the role of IL-12p70, IL 17A or IL33, etc. other cytokines and chemokines and further soluble proteins such as the danger-associated molecular pattern S100A8/A9 have become of an increasing interest over the last years (Slaats et al. 2016, So et al. 2017, Alvarez et al. 2017, Foell et al. 2007). The heterodimeric complex S100A8/A9 consists of the proteins S100A8 and S100A9 and is also called calprotectin (Leukert et al. 2006, Strupat et al. 2000). S100A8/A9 is primarily expressed in neutrophil granulocytes and monocytes but can also be observed in keratinocytes (Ross et al. 2001, Sorenson et al. 2012, Frosch et al. 2000, Ehrchen et al. 2009). Several reports have described S100A/A9 as a potential biomarker in different chronic and acute diseases including diseases of the oropharynx (Frosch et al. 2000, Schlegel et al. 1995, van den Bosch et al. 2016, Tyden et al. 2013, Spiekermann et al. 2016).

However, due to possible harms of surgery such as pain or postoperative hemorrhage and the uncertainty of postoperative persistence of sore throat episodes, indication for tonsillectomy has become a source of controversy over the last years (Burton et al. 2014, Stelter et al. 2014). Diagnosis of RAT is based on clinical signs and history taking but an objective criterion is still missing.

Thus, there is still a need for (an) objective and reliable biomarker(s) to identify patients with an increased risk of recurrent tonsillitis and to specify the patient selection for tonsillectomy or tonsillotomy. Ideally, this biomarker should allow for an early identification or the prediction of an increased risk of recurrent acute tonsillitis in patients without any symptoms of acute tonsillitis. The technical problem therefor is to comply with this need. The technical problem is solved by providing the embodiments reflected in the claims, described in the description and illustrated in the Examples and Figures that follow.

### SUMMARY OF THE INVENTION

The present invention is, at least partly, based on the surprising finding that cytokines/chemokines such as IL-1β, IL-6, IL-18, IL-33, S100A8/S100A9 and TNFα are significantly increased in symptomless patients who underwent or have to undergo a tonsillectomy after a diagnosis of recurrent (episodes of) acute tonsillitis (RAT) in comparison to a healthy control group (Examples 1 and 2, FIGS. 1-3). Surprisingly, the biomarkers IL-1β, IL-18 and S100A8/S100A9 are particularly useful in the diagnosis of RAT, in the prediction of an increased risk or the predisposition for RAT and for stratifying a subject for necessity for tonsillectomy or tonsillotomy (see Examples 1 and 2). As shown in Example 3 and FIG. 4, an increase of at least two of the biomarkers IL-1β, IL-18 and S100A8/S100A9 may be indicative for the diagnosis of RAT, i.e. indicative for the necessity of tonsillectomy or tonsillotomy and also indicative for an elevated risk or a predisposition for RAT.

Accordingly, in one aspect, the present invention provides a method of stratifying a subject having no symptoms of acute tonsillitis for necessity for tonsillectomy or tonsillotomy, comprising: (i) determining the amount of the biomarkers IL-1β, IL-18 and S100A8/S100A9 in a sample obtained from said subject, and (ii) stratifying said subject for the necessity of tonsillectomy or tonsillotomy by comparing the amount of said biomarkers with a threshold value for each biomarker.

In the method of stratifying of the invention, an increased amount of at least two of said biomarkers relative to the respective threshold value is indicative for the necessity for tonsillectomy or tonsillotomy in said subject.

Contrary thereto, in the method of stratifying of the invention, no increased amount of at least two of said biomarkers relative to the respective threshold value indicates that there is no necessity for tonsillotomy or tonsillotomy in said subject.

In another aspect, the present invention relates to a method of diagnosing recurrent tonsillitis in a subject having no symptoms of acute tonsillitis, comprising: ( ) determining the amount of the biomarkers IL-1β, IL-18 and S100A8/S100A9 in a sample obtained from said subject, and (ii) diagnosing recurrent tonsillitis in said subject by comparing the amount of said biomarkers with a threshold value for each biomarker.

In the method of diagnosing recurrent tonsillitis of the invention, an increased amount of at least two of said biomarkers relative to the respective threshold value is indicative for the presence of recurrent tonsillitis in said subject.

Contrary thereto, in the method of diagnosing recurrent tonsillitis of the invention, no increased amount of at least two of said biomarkers relative to the respective threshold value indicates that there is no presence of recurrent tonsillitis in said subject.

In still another aspect, the present invention relates to a method of predicting the risk or the predisposition for recurrence of acute tonsillitis (RAT) in a subject having no symptoms of acute tonsillitis, comprising: (i) determining the amount of the biomarkers IL-1β, IL-18 and S100A8/S100A9 in a sample obtained from said subject, and (ii) predicting the risk or the predisposition for recurrence of acute tonsillitis by comparing the amount of said biomarkers with a threshold value for each biomarker.

In the method of predicting the risk or the predisposition for, an increased amount of at least two of said biomarkers relative to the respective threshold value is indicative for an elevated risk or a predisposition for recurrence of acute tonsillitis.

Contrary thereto, in the method of predicting the risk or the predisposition for, no increased amount of at least two of said biomarkers relative to the respective threshold value indicates no elevated risk or no predisposition for recurrence of acute tonsillitis.

It is envisaged that the subject having no symptoms of acute tonsillitis does not fulfil the criterion of having at least six episodes of diagnosed purulent acute tonsillitis in the previous year, wherein each episode is preferably characterized by administration of antibiotics.

Preferably, the subject having no symptoms of acute tonsillitis fulfils the criterion of having at least six episodes of diagnosed purulent acute tonsillitis in the previous year, wherein each episode is preferably characterized by administration of antibiotics.

Preferably, the acute tonsillitis is a bacterial tonsillitis or viral tonsillitis. More preferably, the acute tonsillitis is bacterial.

Preferably, the sample is a saliva sample, a serum sample, a tissue sample, a blood sample, a urine sample, a lymphatic fluid sample, a nasopharyngeal wash sample, a sputum sample, a mouth swab sample, a throat swab sample, a nasal swab sample, a bronchoalveolar lavage sample, or a bronchial secretion sample.

More preferably, the amount of S100A/S100AB and IL-18 is determined in a serum sample and wherein the amount of IL-1β is determined in a saliva sample.

Preferably, the amount of S100A8/S100A9, IL-1β and IL-18 is determined by using a multiplex bead-based assay, mass spectrometry, chromatography, turbidity assay, lateral flow technology and/or an immunoassay.

Preferably, the threshold value is a predetermined threshold value.

It is envisaged that the threshold value is calculated by comparing the amount of the at least two biomarkers in a group of patients diagnosed with recurrence of acute tonsillitis (RAT) and having no symptoms of acute tonsillitis with the amount of said biomarker in a healthy control group.

Preferably, the patients diagnosed with recurrence of acute tonsillitis (RAT) fulfil the criterion of having at least six episodes of diagnosed purulent acute tonsillitis in the previous year, wherein each episode is preferably characterized by administration of antibiotics.

Preferably, the threshold value is calculated by ROC analysis, wherein the area under the curve (A-value) is at least 0.7, preferably at least 0.8, more preferably at least 0.9.

Preferably, the symptoms of acute tonsillitis are sore throat, red and swollen tonsils and pain when swallowing.

It is envisaged that the subject is a mammal, preferably a human subject.

Preferably, said subject is an adult, a juvenile or a child.

### BRIEF DESCRIPTION OF THE DRAWINGS

FIG. 1: Cytokine levels in saliva of RAT patients compared to healthy patients. Cytokine levels of IL-1ß (A), IFN-α, (B), IFN-γ (C), TNF-α (D), MCP-1 (E), IL-6 (F), IL-8 (G), IL-10 (H), IL-12p70 (I), IL-17 (J), IL-18 (K) and IL-33 (L) in saliva are shown. Levels of IL-1ß, IL-6, TNF-α and IL-33 are significantly increased in patients with recurrent tonsillitis compared to healthy controls. (*p<0.05)

FIG. 2: Cytokine levels in serum of RAT patients compared to healthy patients. Cytokine levels of IL-1ß (A), IFN-α, (B), IFN-γ (C), TNF-α (D), MCP-1 (E), IL-6 (F), IL-8 (G), IL-10 (H), IL-12p70 (I), IL-17 (J), IL-18 (K) and IL-33 (L) in serum are shown. Significantly increased levels of IL-18 could be observed in patients with recurrent tonsillitis compared to healthy controls. (**p<0.01)

FIG. 3: S100A/S100A9 levels in serum and saliva of RAT patients compared to healthy patients. S100A8/A9 levels in serum (A) and saliva (B) of healthy controls and patients with recurrent tonsillitis are shown. Significantly increased levels could be observed in serum of patients with RAT. (*p<0.05)

FIG. 4: Receiver Operating Characteristic curve analysis of the RAT score. A cut-off value of 1.5 could be determined for the RAT score to identify patients with recurrent tonsillitis with a sensitivity of 0.95 and a specificity of 0.88. Area under the curve A=0.959.

### DETAILED DESCRIPTION OF THE INVENTION

Although various reports have been focused on the expression of various cytokines and chemokines in the course of recurrent tonsillitis, the inventors were for the first time able to find an objective criterion to identify patients suffering from recurrent (episodes of) acute tonsillitis (RAT). In particular, by analyzing serum and saliva, increased levels of IL-1β, IL-18 and S100A8/S100A9 could be observed in symptomless patients suffering from RAT compared to healthy controls. These parameters were combined to a so called “RAT-score”, which allows a reliable patient identification with a sensitivity of 95% and a specificity of 88%. Equally, the RAT-score allows for the prediction of an increased risk or the predisposition for recurrent episodes of acute tonsillitis. Hence, the RAT-score represents the first objective criterion to determine the diagnosis of recurrent episodes of acute tonsillitis and improves the patient selection for tonsillectomy and tonsillotomy. Thus, the methods and uses described and provided by the present invention allow for a reduction of illness-related absence from work and a noticeably improved quality of life. In sum, the present invention relates to a method of stratifying a subject having no symptoms of acute tonsillitis for tonsillectomy or tonsillotomy, to a method of diagnosing recurrent tonsillitis in a subject having no symptoms of acute tonsillitis and to a method of predicting the risk or the predisposition for recurrence of acute tonsillitis (RAT) in a subject having no symptoms of acute tonsillitis.

According to a recommendation of Paradise et al. and different guidelines, the current indication for tonsillectomy or tonsillotomy depends on the frequency of bacterial tonsillitis episodes within the last years (Paradise et al. 1984, Windfuhr et al. 2016, Williams at al. 2002).

This approach is however associated with several biases and disadvantages. The diagnosis of tonsillitis is based on physical examination which allows no reliable differentiation between bacterial and viral infection (Bird et al. 2014, Burton et al. 2014, Stelter at al. 2014). Hence, due to the high percentage of viral etiology of acute tonsillitis (50-80% of the cases) and the frequent prescription of antibiotics, a high percentage of incorrect diagnoses is assumable (Bird et al. 2014, Gulliford et al. 2014). In this context, the limited value of the Cantor criteria (cough absent, tonsillar exudates, history of fever, tender anterior cervical adenopathy, age under 15 years or age over 44 years) has to be mentioned which allow an estimation of the probability of an infection with Group A streptococcus but is not eligible for a precise differentiation between bacterial and viral infection (Ebell et al. 2000, Freer at al. 2017). Furthermore, a swap of the exudative tonsillitis is considered not to be rational because most pathogens belong to the healthy flora and positive results can be detected in 40% of asymptomatic carriers (Bird at al. 2014, Stefter at al. 2014). Inconsistent medical documentation or incorrect information from the patients causes a bias in history-taking with an over- or underestimation of the frequency of bacterial tonsillitis episodes (Morad at al. 2017). Due to the vague statements of the patients, the variability in diagnosis of bacterial tonsillitis and the associated doubtful use of antibiotic treatment, the frequency of episodes of bacterial tonsillitis seems to be an ineligible criterion for identification of patients profiting from tonsillectomy.

Several reports have shown the benefits of the tonsillectomy. Patients suffering from recurrent tonsillitis or sore throat in common showed a significant decrease of sore throat episodes, missing-work or school days, a reduced use of medical resources and an increase in quality of life (Mui et al. 1998, Morad et al. 2017, Senska at al. 2015, Douglas et al. 2017, Koskenkorva at al. 2009). These benefits are faced with the possible risks of surgery such as postoperative hemorrhage, general morbidity or a prolonged hospitalization. That implies the need of a novel, objective criterion or diagnostic test for an improved, specified patient selection. The need is fulfilled by the claimed subject matter.

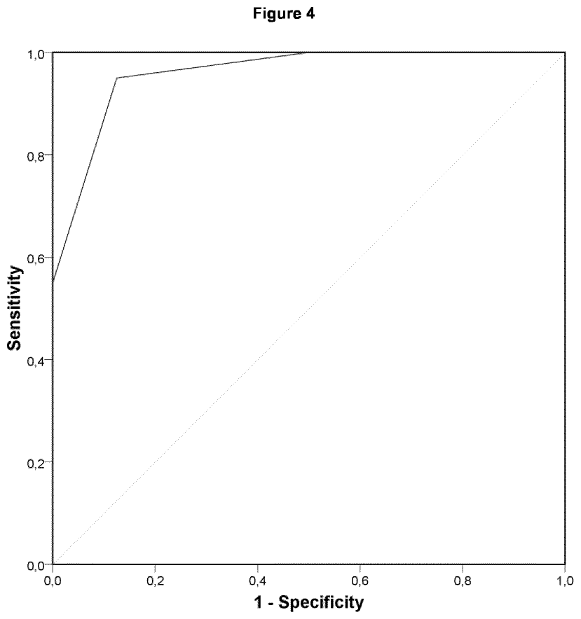

As surprisingly discovered by the inventors of the present invention, elevated levels of IL-18, S100A8/A9 in serum and IL-1ß in saliva can be combined to form the RAT-score as a novel, objective biomarker to identify symptomless patients-suffering from recurrent (episodes of) acute tonsillitis (see Example 3, FIG. 4). In this respect, application of the RAT score allows a better advice and a more specific patient selection for tonsillectomy as well as tonsillotomy. As shown in Example 3, the cut-off value for the RAT score has been determined to be 1.5 points, i.e. at least two of the three biomarkers should be above its respective threshold value to support the necessity for a tonsillectomy or tonsillotomy or a diagnosis of RAT.

Accordingly, the present invention relates to a method of stratifying a subject having no symptoms of acute tonsillitis for necessity for tonsillectomy or tonsillotomy, comprising: (i) determining the amount of the biomarkers IL-1β, IL-18 and S100A8/S100A9 in a sample obtained from said subject, and (ii) stratifying said subject for necessity for tonsillectomy or tonsillotomy by comparing the amount of said biomarkers with a threshold value for each biomarker. As stated herein above, an increased amount of at least two of said three biomarkers relative to the respective threshold value is indicative for the necessity for tonsillectomy or tonsillotomy in said subject, wherein no increased amount of at least two of said three biomarkers relative to the respective threshold value indicates that there is no necessity for tonsillectomy or tonsillotomy in said subject.

The term “indicative for” when used in the context of the methods and uses described herein means that increased amounts of at least two of said three biomarkers in a sample from said subject relative to the respective threshold value are potential risk factors or risk indicators as to whether or not a subject may have an increased risk for recurrent (episodes of) acute tonsillitis or a decreased or no risk recurrent (episodes of) acute tonsillitis, respectively. Thus, the amounts of the biomarkers IL-1β, IL-18 and S100A8/S100A9 in a sample from a subject are, so to say, risk-stratification biomarkers. Hence, based on the findings of the present inventors, it is possible to apply a risk management for subjects for developing recurrent (episodes of) acute tonsillitis, with the aim of an early identification of patients with the necessity for tonsillectomy or tonsillotomy.

Interleukin 1 beta (IL-1β), also known as leukocytic pyrogen, leukocytic endogenous mediator, mononuclear cell factor or lymphocyte activating factor, is a member of the interleukin 1 family of cytokines. This cytokine is an important mediator of the inflammatory response, and is involved in a variety of cellular activities, including cell proliferation, differentiation, and apoptosis. The UniProt Accession number of human IL-1β is P01584 and its HUGO number is HGNC:5992.

Interleukin 18 (IL-18), also known as interferon-gamma inducing factor, is a member of the interleukin 1 family of cytokines. IL-18 works by binding to the interleukin-18 receptor, and together with IL-12 it induces cell-mediated immunity following infection with microbial products like lipopolysaccharide (LPS). After stimulation with IL-18, natural killer (NK) cells and certain T cells release another important cytokine called interferon-γ (IFN-γ or IFNG) or type II interferon that plays an important role in activating the macrophages or other cells. The UniProt Accession number of human IL-18 is Q14116 and its HUGO number is HGNC:5986.

The methods and uses described herein refer to the determination of the amount of S100A8/S100A9 in a sample. Preferably, when measuring the amount or concentration of S100A8/S100A9, said measurement relates to the amount of the heterodimeric complex of S100A8/S100A9. However, also the concentration of the S100AB and/or the S100A9 homodimer can be equally determined in this respect. The S100A8/S100A9 complex is also known as calprotectin. In the presence of calcium, S100A8/S100A9 (calprotectin) is capable of sequestering the transition metals manganese and zinc via chelation. This metal sequestration affords the complex antimicrobial properties. S100A8/S100A9 (calprotectin) comprises as much as 60% of the soluble protein content of the cytosol of a neutrophil, and it is secreted by an unknown mechanism during inflammation. The UniProt Accession number of human S100A8 is P05109 and its HUGO number is HGNC: 10498. The UniProt Accession number of human S100A9 is P06702 and its HUGO number is HGNC:10499.

In the method of stratifying a subject having no symptoms of acute tonsillitis for necessity for tonsillectomy or tonsillotomy, an increased amount of at least two of said biomarkers relative to the respective threshold value may be indicative for the necessity for tonsillectomy or tonsillotomy in said subject. In a specific embodiment, an increased amount of three of said biomarkers relative to the respective threshold value is indicative for the necessity for tonsillectomy or tonsillotomy in said subject. Vice versa, no increased amount of at least two of said biomarkers relative to the respective threshold value indicates that there is no necessity for tonsillectomy or tonsillotomy in said subject in the method of stratifying a subject having no symptoms of acute tonsillitis for necessity for tonsillectomy or tonsillotomy.

“Stratifying” as used herein, relates to a process of categorizing or classifying of a subject. For example, if a subject has an increased amount above a threshold value for at least two biomarkers of the invention, the subject is “stratified” into a group of subjects that should undergo tonsillectomy or tonsillotomy. In the opposite case, e.g., a subject that does not have an increased amount of at least two biomarkers in comparison to a threshold value, the subject is “stratified” into a group of subjects that should not undergo tonsillectomy or tonsillotomy. In this context, “necessity for tonsillectomy or tonsillotomy” may relate to the stratification into a group of subjects that should undergo tonsillectomy or tonsillotomy.

In the method and uses of the invention, the amount of the biomarkers may be compared with a respective threshold value for each single biomarker. “Comparing the amount” of biomarkers from a sample of a subject with a threshold value when used herein means that said sample can be compared to a (predetermined) threshold value in any suitable manner. If the amount of a biomarker is higher than the respective threshold for this biomarker, the amount of the biomarker can be described as increased. If the amount of a biomarker is equal or lower than the respective threshold value for this single biomarker, the amount of the biomarker can be described as not increased.

A “threshold value” or “cutoff value” as used herein relates to a certain value or limit for each of the biomarkers of the invention, in particular IL-1β, IL-18 and S100A8/S100A9, or any additional biomarker described herein. This threshold value can be understood as a numerical or quantifiable differentiator that allows a decision whether a determined value of a biomarker is within the range of a healthy subject or group of healthy subjects or is increased in comparison to a healthy subject or a group of healthy subjects, i.e. the threshold value may be a binary classifier. The term “group of healthy subject” as used herein can also be replaced by the term “control group”. The threshold value is individual for each biomarker and/or additional biomarker.

This threshold value may be a predetermined threshold value. Suitable methods for the (pre-)determination of a threshold value are known to a person skilled in the art. The determination of the threshold value may be based on or be calculated by comparing the amount of the biomarkers in a group of patients already diagnosed with recurrence of acute tonsillitis (RAT), but having no symptoms of acute tonsillitis, with the amount of said biomarker in a healthy control group. The group, which is diagnosed with RAT and has no symptoms of acute tonsillitis at the moment of measurement, may fulfil the criterion of having at least six episodes of diagnosed purulent acute tonsillitis in the previous year, wherein each episode is preferably characterized by administration of antibiotics.

Threshold values of IL-1β, IL-18 and S100A8/S100A9 may change with different detection methods for IL-1β, IL-18 and S100A8/S100A9. Hence, the individual threshold values have to be determined for the specific detection method used when applying the methods or uses of the present invention. A person skilled in the art can readily determine the exact threshold values for a specific set of analysis methods. Usually, a group of healthy subjects and a group of symptomless subjects suffering from RAT, are analyzed for the amount of the respective biomarker. The threshold value may then be set to a value that allows the discrimination between healthy and symptomless subjects suffering from RAT with highest precision, i.e. to a value that is optimized for highest specificity (true negative rate) and highest amount of sensitivity (true positive rate).

One exemplary method applied for determining a respective threshold value uses the receiver operating characteristic (ROC) curve for optimization of threshold values. A ROC curve is a graphical plot that illustrates the diagnostic ability of a binary classifier system as its discrimination threshold is varied. The ROC curve is created by plotting the true positive rate (TPR) against the false positive rate (FPR) at various threshold settings. The true-positive rate is also known as sensitivity. The false-positive rate is also known as the fall-out or probability of false alarm and can be calculated as (1—specificity). It can also be thought of as a plot of the Power as a function of the Type I Error of the decision rule. The ROC curve is thus the sensitivity as a function of fall-out (1—specificity). In general, if the probability distributions for both detection/recall and false alarm are known, the ROC curve can be generated by plotting the cumulative distribution function (area under the probability distribution from −∞ to the discrimination threshold) of the detection probability in the y-axis versus the cumulative distribution function of the false-alarm probability on the x-axis. An exemplary method to determine optimized threshold values using the ROC curve or plots is discussed in Zweig et al. (1993), Clin Chem, 39(4):581-577, which is incorporated herein by reference. For determination of optimized threshold values, the software SPSS from IBM may be used for ROC analysis. The quality of threshold values is expressed as the area under the curve, the so-called A-value.

Accordingly, the threshold value, which has been determined and is applied according to the methods and uses of the present invention, preferably has an area under the curve (A-value) of at least 0.7, at least 0.8, at least 0.9 or at least 0.95.

If the assays as described in the Examples 1 and 2 are used, the determination of new threshold values may not be necessary. These Examples refer to the use of cytokine/chemokine analysis methods as described in the Examples 1 and 2, i.e. the LEGENDplex™ assay “Human Inflammation Panel” of BioLegend for IL-1β and IL-18 and an ELISA for S100A8/S100A9 as described in Frosch et al. (2000). Accordingly, in view of the present invention, the threshold value for S100A8/S100A9 in serum may correspond to a threshold value of 757 pg/ml as determined with an ELISA for S100AB/S100A9 as described in Frosch et al. (2000). Similarly, the threshold value for IL-1β in saliva may correspond to a threshold value of 30 pg/ml as determined with LEGENDplex™ assay “Human Inflammation Panel” of BioLegend and the threshold value for IL-18 in serum may correspond to a threshold value of 44 pg/ml as determined with LEGENDplex™ assay “Human Inflammation Panel” of BioLegend. According to the Examples of the present invention, it is envisaged that the threshold value for S100A8/A9 in serum has a sensitivity of at least 0.55 and a specificity of at least 0.85, preferably when determined with an ELISA such as the ELISA described in Frosch et al. (2000). Similarly it is envisaged that the threshold value for IL-1β in serum has a sensitivity of at least 0.65 and a specificity of at least 0.85, preferably when determined with a multiplex bead-based assay such as the LEGENDplex™ assay “Human Inflammation Panel” of BioLegend, and the threshold value for IL-18 in serum has a sensitivity of at least 0.9 and a specificity of at least 0.6, preferably when determined with a multiplex bead-based assay such as the LEGENDplex™ assay “Human Inflammation Panel” of BioLegend.

The term “tonsillitis” as used herein relates to a painful and purulent inflammation of the tonsils. Generally, tonsillitis can be further subdivided into acute tonsillitis, chronic tonsillitis and recurrent acute tonsillitis (RAT). “Acute tonsillitis” describes an episode of tonsillitis that typically remits within about 2 weeks. Symptoms of acute tonsillitis include sore throat, red and swollen tonsils and pain when swallowing. “Chronic tonsillitis” is characterized by a tonsillitis, which symptoms persist for at least two, at least three, at least four or at least five weeks. Both, infection with a bacterium, which is antibiotic resistant, or altered immunologic function, may play a role in the development of chronic tonsillitis. Sleep apnea is a serious complication of chronic tonsillitis and is often an indication for tonsillectomy or tonsillotomy.

In contrast thereto, the present invention focuses on “recurrent acute tonsillitis” (RAT), characterized by a repeated, i.e. periodically re-occurrence of acute tonsillitis. Infections causing RAT may initially respond to antibiotic treatment, but normally return on a frequent basis. A diagnosis of RAT is usually an indication for a tonsillectomy or tonsillotomy. One important decision criterion for the diagnosis of RAT as used in the art is the criterion of having at least six episodes of diagnosed purulent acute tonsillitis in the previous year, wherein each episode is preferably characterized by administration of antibiotics. This criterion is published in the guideline “S2k-Leitlinie 017/024: Therapie entzündlicher Erkrankungen der Gaumenmandeln” published by AWMF online and created by Deutsche Gesellschaft for Hals-Nasen-Ohren-Heilkunde, Kopf-und Hals-Chirurgie; Deutscher Berufsverband der Hals-Nasen-Ohrenärzte; Deutsche Gesellschaft for Kinder-und Jugendmedizin and Deutsche Gesellschaft for Pädiatrische Infektiologie. However, since tonsillitis is often accompanied by severe disease symptoms, an early diagnosis of recurrence is highly beneficial for the patient.

Each episode of acute tonsillitis may be caused by a bacterial or viral infection. Whereas the usual criteria for the indication of tonsillectomy or tonsillotomy concentrate on bacterial infections, a differentiation between bacterial and viral infections is very often difficult or impossible without excessive additional diagnosis. Such additional diagnosis may include PCR, qPCR, ELISA, Western Blot, sequencing, FISH and many more. The methods and uses described herein may refer to the diagnosis of recurrent acute tonsillitis caused by bacterial infections. Typical bacteria causing tonsillitis include, but are not limited to, group β-hemolytic streptococcus, Staphylococcus aureus (including methicillin resistant Staphylococcus aureus or MRSA), Streptococcus pneumoniae, Mycoplasma pneumoniae, Chlamydia pneumoniae, Bordetella pertussis, Fusobacterium sp., Corynebacterium diphtheriae, Treponema pallidum, and Neisseda gonorrhoeae. In a preferred embodiment, the acute tonsillitis is caused by an infection with group A-hemolytic streptococcus. However, the present invention is not limited to bacterial infections but can be applied regardless of the nature of the cause of the tonsillitis.

Thus, the recurrent acute tonsillitis may also be caused by viral infections. Typical viruses causing tonsillitis include, but are not limited to, adenovirus, rhinovirus, influenza, coronavirus, and respiratory syncytial virus, Epstein-Barr virus, herpes simplex virus, cytomegalovirus, or HIV.

During an episode of acute bacterial or viral tonsillitis, the amount of inflammation markers, such as the biomarkers IL-1β, IL-18 and S100A8/S100A9, is drastically increased.

Hence, it is not possible to apply the methods and uses of the invention in a subject, which currently suffers from acute tonsillitis. The present invention is only intended to be used in subjects having no symptoms of acute tonsillitis, but may or may not suffer from RAT. Hence, a subject showing symptoms of acute tonsillitis, such as sore throat, red and swollen tonsils and pain when swallowing, cannot be diagnosed, stratified or risk-assessed using the methods of the invention. However, this does not exclude the possibility that a subject can be tested after remission of an episode of acute tonsillitis. In contrast, the methods and uses of the invention may be applied even after the very first acute tonsillitis, i.e. in patients without a long history of relapsing tonsillitis. In this case, further unnecessary pain and suffering could be avoided, which would be caused by waiting until the required number for a diagnosis of RAT would be fulfilled.

“Tonsillectomy” as used herein is a surgical procedure in which both palatine tonsils (hereafter called “tonsils”) are removed from a recess in the side of the pharynx called the tonsillar fossa. The procedure usually is performed in response to recurrence of acute tonsillitis, sleep surgery for obstructive sleep apnea, nasal airway obstruction, diphtheria carrier state, snoring, or peritonsillar abscess. For children, tonsillectomy is usually combined with an adenoidectomy, which is the removal of the adenoid (also known as the pharyngeal tonsil or nasopharyngeal tonsil). Although tonsillectomy is nowadays performed much less frequently than in the 1950s, it remains a common surgical procedure in children in the United States and many other western countries. “Tonsillotomy” (also known as intracapsular, subtotal or partial tonsillectomy) as used herein is a surgical procedure, in which only a portion of the tonsils is removed. Complications after a partial tonsillectomy are similar to those of a total tonsillectomy (i.e. postoperative pain, hemorrhage, decreased oral intake).

The sample, in which the amount of the biomarkers is determined according to the present invention, may be any sample obtained from the symptomless subject of the present invention and potentially comprises the biomarkers IL-1β, IL-18 and S100A8/S100A9 and/or any additional biomarker described herein. The term “sample”, when used herein, relates to a material or mixture of materials, typically but not necessarily in liquid form, containing one or more biomarkers of the invention. Preferably, the sample of the present invention is a biological sample. The term “biological sample” as used herein refers to a sample obtained from a subject, wherein the sample may be any biological tissue or fluid sample. Frequently, the sample will be a “clinical sample” which is a sample derived from a patient. Preferably, the biological sample of the present invention is a serum sample, a plasma sample, a urine sample, a feces sample, a saliva sample, a tear fluid sample, or a tissue extract sample. Further samples envisaged are sputum, cerebrospinal fluid, fine needle biopsy samples, peritoneal fluid, and pleural fluid, but the invention is not limited thereto. In the context of the present invention, the sample may be a saliva sample, a serum sample, a tissue sample, a blood sample, a urine sample, a lymphatic fluid sample, a nasopharyngeal wash sample, a sputum sample, a mouth swab sample, a throat swab sample, a nasal swab sample, a bronchoalveolar lavage sample, or a bronchial secretion sample. Biological samples may also include sections of tissues such as frozen sections taken for histological purposes. Such samples include, for example, whole blood, serum, etc.

Preferably, a sample is a sample that includes extracellular material or liquid. Biological samples can be analyzed directly or they may be subject to some preparation prior to use in methods or kits of the present invention. Such preparation can include, but is not limited to, suspension/dilution of the sample in water or an appropriate buffer or removal of cellular debris, e.g. by centrifugation, or selection of particular fractions of the sample before analysis.

Preferably, the sample is a saliva sample, a serum sample, a tissue sample, a blood sample, a urine sample, a lymphatic fluid sample, a nasopharyngeal wash sample, a sputum sample, a mouth swab sample, a throat swab sample, a nasal swab sample, a bronchoalveolar lavage sample, or a bronchial secretion sample. In a preferred embodiment, the amount of S100A8/S100A9 and IL-18 is determined in a serum sample and the amount of IL-1β is determined in a saliva sample.

The subject of the present invention is preferably a mammal. The mammal may be any one of mouse, rat, guineas pig, rabbit, cat, dog, monkey, horse, or human. Preferably, the subject is a human. In a specific embodiment, the human is an adult. The subject may be a patient suffering from RAT or being at risk to develop RAT. In a further specific embodiment, the human tested according to the methods and uses of the invention is a child having no symptoms of acute tonsillitis. In another specific embodiment, the human is a juvenile having no symptoms of acute tonsillitis. An adult as used herein relates to a human, which is at least 18 years old. A child relates to a human, which is 14 years or younger. A juvenile as used herein relates to a human, which is between 14 and 18 years old. In another embodiment, the subject is a human younger than 18 years. The methods and uses of the present invention are equally applicable to all groups of patients described herein above. As explained elsewhere herein, a sample may be analyzed that has been obtained from said subject, which is typically a living organism. Where the subject is a living human who may receive treatment or diagnosis for a disease or condition as described herein, it is also addressed as a “patient”. In other embodiment, the subject may be an animal under the proviso that said animal has tonsils. The animal may be a dog, a cat, a cow, a pig, a sheep or a goat.

The current diagnosis of RAT described in the art and the corresponding decision for or against a tonsillectomy or tonsillotomy is based on the previous history of tonsillitis episodes. However, the previous history is often insufficiently documented and biased. On the other hand, a subject could profit from a tonsillectomy or tonsillotomy even if the current standard criteria for an indication to perform a tonsillectomy or tonsillotomy have not yet been fulfilled. Thus, the present invention may also be useful to make a decision whether or not a tonsillectomy or tonsillotomy is indicated before the subject suffered many episodes of tonsillitis resulting in unnecessary pain and illness. Hence, the invention may be used to make an early decision and to avoid the so-called “watchful waiting”. Accordingly, the subject may not fulfil the criterion of having at least six episodes of diagnosed purulent acute tonsillitis in the previous year, wherein each episode is preferably characterized by administration of antibiotics. This criterion was published in the guideline “S2k-Leitlinie 017/024: Therapie entzündlicher Erkrankungen der Gaumenmandeln” published by AWMF online and created by Deutsche Gesellschaft für Hals-Nasen-Ohren-Heilkunde, Kopf-und Hals-Chirurgie; Deutscher Berufsverband der Hals-Nasen-Ohrenärzte; Deutsche Gesellschaft für Kinder-und Jugendmedizin and Deutsche Gesellschaft für Pädiatrische Infektiologie.

However, the invention may of course also be suitable for subjects who fulfil this criterion and may here support the diagnosis of RAT, the decision about the necessity of tonsillectomy or tonsillotomy or risk-assessment. Accordingly, the subject having no symptoms of acute tonsillitis may fulfil the criterion of having at least six episodes of diagnosed purulent acute tonsillitis in the previous year, wherein each episode is preferably characterized by administration of antibiotics.

“Determining” or “quantifying” the amount of the biomarkers, particularly IL-1β, IL-18 and/or S100A/S100A9, can be carried out by way of any suitable technique available and known to those skilled in the art. In some embodiments, determining the amount of the biomarkers in a biological sample comprises the use of mass spectrometry. Mass spectrometry works by ionizing chemical compounds to generate charged molecules or molecule fragments and measuring their mass-to-charge ratios. In this regard, spectra are used to determine the elemental or isotopic signature of a sample, the masses of particles and of molecules, and to elucidate the chemical structures of molecules. In some embodiments, determining the amount of the biomarkers in a biological sample comprises the use of aptmer-target-binding technology. When applying aptamer-target-binding technology, the biomarkers are identified by a class of small nucleic acid ligands (aptamers). In some embodiments the aptamers are composed of RNA having high specificity and affinity for their targets. In some embodiments the aptamers are composed of single-stranded DNA oligonucleotides having high specificity and affinity for their targets. Similar to antibodies, aptamers interact with their targets by recognizing a specific three-dimensional structure and are thus termed “chemical antibodies.” In contrast to protein antibodies, aptamers offer unique chemical and biological characteristics based on their oligonucleotide properties.

In other embodiments “determining” or “quantifying” the amount of the biomarkers, particularly IL-1β, IL-18 and/or S100A8/S100A9, and/or any other additional biomarker described herein, in a sample comprises the use of an immunoglobulin having binding specificity to the biomarkers. Examples of suitable immunoassay techniques in this regard are radiolabel assays such as a Radioimmunoassay (RIA) or enzyme-immunoassay such as an Enzyme Linked Immunosorbent Assay (ELISA), Luminex®-assays, precipitation (particularly immunoprecipitation), a sandwich enzyme immune test, an electro-chemiluminescence sandwich immunoassay (ECLIA), a dissociation-enhanced lanthanide fluoro immuno assay (DELFIA), a scintillation proximity assay (SPA), turbidimetry, nephelometry, latex-enhanced turbidimetry or nephelometry, or a solid phase immune test. Further methods known in the art (such as gel electrophoresis, 2D gel electrophoresis, SDS polyacrylamide gel electrophoresis (SDS-PAGE), and Western Blotting, can be used alone or in combination with labelling or other detection methods as described herein.

Within the context of the present invention, an ELISA or RIA test can be competitive for measuring the amount of the biomarkers, particularly IL-1β, IL-18 and/or S100A8/S100A9, and/or any other additional biomarker described herein, i.e. the amount of antigen. For example, an enzyme labelled antigen is mixed with a test sample containing antigen, which competes for a limited amount of immunoglobulin. The reacted (bound) antigen is then separated from the free material, and its enzyme activity is estimated by addition of substrate. An alternative method for antigen measurement is the double immunoglobulin sandwich technique. In this modification a solid phase is coated with specific immunoglobulin. This is then reacted with the sample from the subject that contains the antigen. Then enzyme labelled specific immunoglobulin is added, followed by the enzyme substrate. The ‘antigen’ in the test sample is thereby ‘captured’ and immobilized on to the sensitized solid phase where it can itself then immobilize the enzyme labelled immunoglobulin. This technique is analogous to the immunoradiometric assays. In an indirect ELISA method, an antigen is immobilized by passive adsorption on to the solid phase. A test serum may then be incubated with the solid phase and any immunoglobulin in the test serum forms a complex with the antigen on the solid phase.

Similarly a solution of a proteinaceous binding molecule with immunoglobulin-like functions may be incubated with the solid phase to allow the formation of a complex between the antigen on the solid phase and the proteinaceous binding molecule. After washing to remove unreacted serum components an anti-immunoglobulin immunoglobulin anti-proteinaceous binding molecule immunoglobulin, linked to an enzyme is contacted with the solid phase and incubated.

Where the second reagent is selected to be a proteinaceous binding molecule with immunoglobulin-like functions, a respective proteinaceous binding molecule that specifically binds to the proteinaceous binding molecule or the immunoglobulin directed against the antigen is used. A complex of the second proteinaceous binding molecule or immunoglobulin and the first proteinaceous binding molecule or immunoglobulin, bound to the antigen, is formed.

Washing again removes unreacted material. In the case of RIA radioactivity signals are being detected. In the case of ELISA the enzyme substrate is added. Its colour change will be a measure of the amount of the immobilized complex involving the antigen, which is proportional to the antibody level in the test sample.

Luminex®-assays in the context of the present invention are designed to simultaneously measure multiple protein targets, e.g. the biomarkers IL-1β and IL-18 in a single sample as described in Example 1. In some embodiment the Luminex®-assay may be designed in a polystyrene format. In some embodiment the Luminex®-assay may be designed in a magnetic bead format. In some embodiments the Luminex®-assay is a multiplex assays.

Further exemplary methods for the detection of the biomarkers IL-1β, IL-18 and/or S100A8/S100A9 include a multiplex bead-based assay, mass spectrometry, chromatography, turbidity assay, lateral flow technology and/or an immunoassay. Exemplary methods for the detection of IL-1β and IL-18 are disclosed in Example 1. An exemplary method of detection of S100A8/S100A9 is disclosed in Example 2.

The invention is not only suitable to make a decision whether a tonsillectomy or tonsillotomy is necessary. A decision for a tonsillectomy or tonsillotomy is usually correlated with a diagnosis of recurrent tonsillitis. Thus, a positive decision for a tonsillectomy or tonsillotomy and a diagnosis of RAT may go hand in hand. Accordingly, the present invention also relates to a method of diagnosing recurrent tonsillitis in a subject having no symptoms of acute tonsillitis, comprising: (i) determining the amount of the biomarkers IL-1β, IL-18 and S100A8/S100A9 in a sample obtained from said subject, and (ii) diagnosing recurrent tonsillitis in said subject by comparing the amount of said biomarkers with a threshold value for each biomarker.

The term “diagnosing” or “diagnosis” when used herein means determining or detecting if a subject suffers from RAT. In view of the present invention, said subject is a symptomless subject. Accordingly, in the method of diagnosing recurrent tonsillitis in a subject having no symptoms of acute tonsillitis, an increased amount of at least two of the biomarkers IL-1β, IL-18 and S100A8/S100A9 relative to the respective threshold value may be indicative for the presence of recurrent tonsillitis in said subject. In a specific embodiment, an increased amount of all three biomarkers relative to the respective threshold value is indicative for the presence of recurrent tonsillitis in said subject. Vice versa, no increased amount of at least two of the biomarkers IL-1β, IL-18 and S100A8/S100A9 relative to the respective threshold value indicates that there is no presence of recurrent tonsillitis in said subject in the method of diagnosing recurrent tonsillitis in a subject having no symptoms of acute tonsillitis.

In addition, the invention is also suitable for predicting the risk or predisposition for recurrence of acute tonsillitis (RAT), i.e. allows for an early assessment as to whether or not a subject is in the physiological/pathological condition to suffer from recurrent episodes of acute tonsillitis in the near future. Accordingly, the present invention further relates to a method of predicting the risk or the predisposition for recurrence of acute tonsillitis (RAT) in a subject having no symptoms of acute tonsillitis, comprising: (i) determining the amount of the biomarkers IL-1β, IL-18 and S100A8/S100A9 in a sample obtained from said subject, and (ii) predicting the risk or the predisposition for recurrence of acute tonsillitis by comparing the amount of said biomarkers with a threshold value for each biomarker.

In the method of predicting the risk or the predisposition for recurrence of acute tonsillitis (RAT) in a subject having no symptoms of acute tonsillitis, an increased amount of at least two of said biomarkers relative to the respective threshold value may be indicative for an elevated risk or a predisposition for recurrence of acute tonsillitis. In a specific embodiment, an increased amount of three of said biomarkers relative to the respective threshold value is indicative for an elevated risk or a predisposition for recurrence of acute tonsillitis. Vice versa, no increased amount of at least two of said biomarkers relative to the respective threshold value may indicate no elevated risk or no predisposition for recurrence of acute tonsillitis in the method of predicting the risk or the predisposition for recurrence of acute tonsillitis (RAT) in a subject having no symptoms of acute tonsillitis.

The term “predict the risk” as used herein relates to the likelihood that a subject who does not currently have clinically apparent RAT will suffer from RAT, i.e. describes the probability of developing RAT. Similarly, the term “predisposition” as used herein relates to a potential or probability for developing RAT in a subject who does not currently have clinically apparent RAT. A subject having increased values of the respective biomarkers will more likely have an increased risk or as predisposition to develop RAT. Accordingly, an increased amount of at least two of the three determined biomarkers may indicate that the subject has a probability of at least 50%, such as at least 60%, at least 70%, at least 80%, at least 90% or at least 95% for developing RAT. Further, no increased amount of at least two of the three determined biomarkers may indicate that the subject has a probability of less than 50%, such as less than 40%, less than 30%, less than 20%, less than 10%, less than 5% or less than 1% for developing RAT. It will be understood by those skilled in the art, that such an assessment on the risk or predisposition is usually not intended to be correct for 100%. However, the term “predicting the risk or predisposition” allows that an estimation can be made for a significantly increased risk for developing recurrent episodes of acute tonsillitis, based on the amount/value of the biomarkers IL-1β, IL-18 and S100A8/S100A9 in a sample obtained from said subject. Whether a value is statistically significant for predicting the risk or predisposition for RAT can be determined by those skilled in the art using various well known statistic evaluation tools, e.g. determination of confidence intervals, p-value determination, Student's t-test, and Mann-Whitney test. Suitable confidence intervals are, at least 90%, at least 95%, at least 97%, at least 98%, or at least 99%. Suitable p-values are preferably 0.1, 0.05, 0.01, 0.005, or 0.0001.

As shown in Example 1, IL-6, IL-33 and TNFα were also significantly increased in patients diagnosed with RAT. However, ROC analysis of IL-6, IL-33 and TNFα concentrations showed that they are no appropriate biomarkers. However, they may be used in addition to the biomarkers IL-1β, IL-18 and S100A8/S100A9 to further improved the diagnostic value.

Accordingly, the method of stratifying a subject having no symptoms of acute tonsillitis may further comprise: (iii) determining the amount of at least one additional biomarker selected from the group consisting of IL-6, IL-33 and TNFα and (iv) stratifying said subject for the necessity of tonsillectomy or tonsillotomy by comparing the amount of said additional biomarkers with a threshold value for each additional biomarker.

Equally, the method of diagnosing recurrent tonsillitis in a subject having no symptoms of acute tonsillitis may further comprise: (iii) determining the amount of the additional biomarkers IL-6, IL-33 and TNFα in a sample obtained from said subject, and (iv) diagnosing recurrent tonsillitis in said subject by comparing the amount of said additional biomarkers with a threshold value for each biomarker.

Equally, the method of predicting the risk or the predisposition for recurrence of acute tonsillitis (RAT) in a subject having no symptoms of acute tonsillitis may further comprise: (i) determining the amount of the additional biomarkers IL-6, IL-33 and TNFα in a sample obtained from said subject, and (ii) predicting the risk or the predisposition for recurrence of acute tonsillitis by comparing the amount of said additional biomarkers with a threshold value for each biomarker.

The present invention further provides for the use of a kit comprising the means for detecting the biomarkers of the present invention for predicting the risk or the predisposition for recurrence of acute tonsillitis (RAT) in a subject having no symptoms of acute tonsillitis, comprising (i) determining the amount of the biomarkers IL-1β, IL-18 and S100A8/S100A9 in a sample obtained from said subject, and (ii) predicting the risk or the predisposition for recurrence of acute tonsillitis by comparing the amount of said biomarkers with a threshold value for each biomarker, wherein an increased amount of at least two of said biomarkers relative to the respective threshold value is indicative for an elevated risk or a predisposition for recurrence of acute tonsillitis. Contrary thereto, no increased amount of at least two of said biomarkers relative to the respective threshold value indicates no elevated risk or no predisposition for recurrence of acute tonsillitis.

Term “kit” when used herein refers to an assembly of useful compounds and other means like solid support plates or test stripes for detecting the biomarkers or additional biomarkers in a sample. Other components such as buffers, controls, and the like, known to those skilled in art, may be included in such test kits. The relative amounts of the various reagents can be varied, to provide for concentrations in solution of the reagents that substantially optimize the sensitivity of the assay. Particularly, the reagents can be provided as dry powders, usually lyophilized, which on dissolution will provide for a reagent solution having the appropriate concentrations for combining with a sample. The present kit may further include instructions for carrying out one or more methods of the present invention, including instructions for using standard and/or composition of the present invention that is included with the kit. In some embodiments the diagnostic kit comprises monoclonal antibodies binding to the biomarkers of the invention. The antibodies used in said kit can be present in bound or soluble from.

The present invention further provides for a kit comprising the means for detecting the biomarkers of the present invention for diagnosing recurrent tonsillitis in a subject having no symptoms of acute tonsillitis, comprising (i) determining the amount of the biomarkers IL-1β, IL-18 and S100A8/S100A9 in a sample obtained from said subject, and (ii) diagnosing recurrent tonsillitis in said subject by comparing the amount of said biomarkers with a threshold value for each biomarker, wherein an increased amount of at least two of said biomarkers relative to the respective threshold value is indicative for the presence of recurrent tonsillitis in said subject.

Contrary thereto, no increased amount of at least two of said biomarkers relative to the respective threshold value indicates that there is no presence of recurrent tonsillitis in said subject.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term ‘containing’ or “including” or sometimes when used herein with the term “having”. When used herein “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be had from the following examples, offered for illustrative purposes only. The examples are not intended to limit the scope of the present invention in any way.

### EXAMPLES

### Materials and Methods

### Study Population

71 Patients who underwent tonsillectomy because of recurrent tonsillitis were included into this study. Study enrolment and surgery was performed in actually asymptomatic patients during the interval without acute tonsillitis. The study population showed a median age of 24 years with a range from 13 to 59 years and consisted of 33 male and 38 female patients corresponding with a gender-ratio of 0.86:1. In the preceding three years the patients had 11.6±5.9 (mean±SD) episodes of recurrent tonsillitis. Healthy volunteers without any history of recurrent tonsillitis or previous tonsillectomy or tonsillotomy (n=15) and a median age of 30 years (ranging from 26 to 59 years) served as controls.

### Ethical Approval

This study was performed in the Department of Otorhinolaryngology, Head and Neck Surgery, University Hospital Münster in accordance with ethical principles, including the World Medical Association Declaration of Helsinki (version 2002) and the additional requirements and has been approved by the institutional ethics committee [2015-217-f-S]. Written informed consent was obtained from all subjects.

### Acquisition of Sera and Saliva Samples

Serum samples were allowed to clot and centrifuged at 2000 g for 10 minutes within 2 hours after acquisition. Saliva sample acquisition was performed with untreated Salivette® (Sarstedt, 51.1534) as described in the manufacturer's datasheet or by collecting saliva in a 50 ml Falcon tube and centrifugation at 1000 g for 15 minutes. Supernatants were aliquoted and stored at −20° C. until analysis.

### Analysis of Chemokines, Cytokines and Soluble Proteins

Quantification of cytokines/chemokines in serum and saliva was performed with the LEGENDplex™ assay “Human Inflammation Panel” (BioLegend) as described in the manufacturer's manual. The “Human Inflammation Panel” allows simultaneous measurement of IL-1ß, IL-6, IL8, IL-1β, IL-12p70, IL17A, IL-18, IL-33, IFN-α, IFN-γ, MCP-1 and TNF-α. Fluorescent signal intensities were detected by NAVIOS™ Flow Cytometer (Beckmann Coulter). S100A8/A9 concentrations were measured with a sandwich enzyme-linked immunosorbent assay (ELISA) for human S100A8/A9 as described earlier (Frosch et al. 2000).

### Statistical Analysis

Statistical analyses were performed with IBM SPSS Statistics 24 and SigmaPlot® 12. Results are described as mean values ±standard error of the mean (mean±SEM) or mean value ±standard deviation (mean±SD) as indicated in the figures. Correlations between variables were determined by Pearson's correlation coefficients (rₚ) and were considered to be low (0.2<rₚ≤0.5), good (0.5<rₚ≤0.8) or excellent (0.8<rₚ≤1.0). Student t-test was used to detect significant differences in parametric results and Mann-Whitney U test was performed to analyze differences between non-parametric groups. Furthermore, Kruskal-Wallis test was used to reveal differences between more than two non-parametric groups. P-values ≥0.05 are considered not to be significant. Significant results are marked with asterisks (*p<0.05, **p<0.01, ***p<0.001). The capacity of the model to differentiate between positive and negative results is described by area under the curve values (A-values) and illustrated by ROC curves which allow calculation of cut-off values. Discriminative power of the model is considered to be excellent with an A-value of ≥0.9, good ≥0.8, and acceptable ≥0.7. A-values <0.7 were considered not to be sufficient for a potential biomarker.

### Example 1: Cytokine and Chemokine Expression

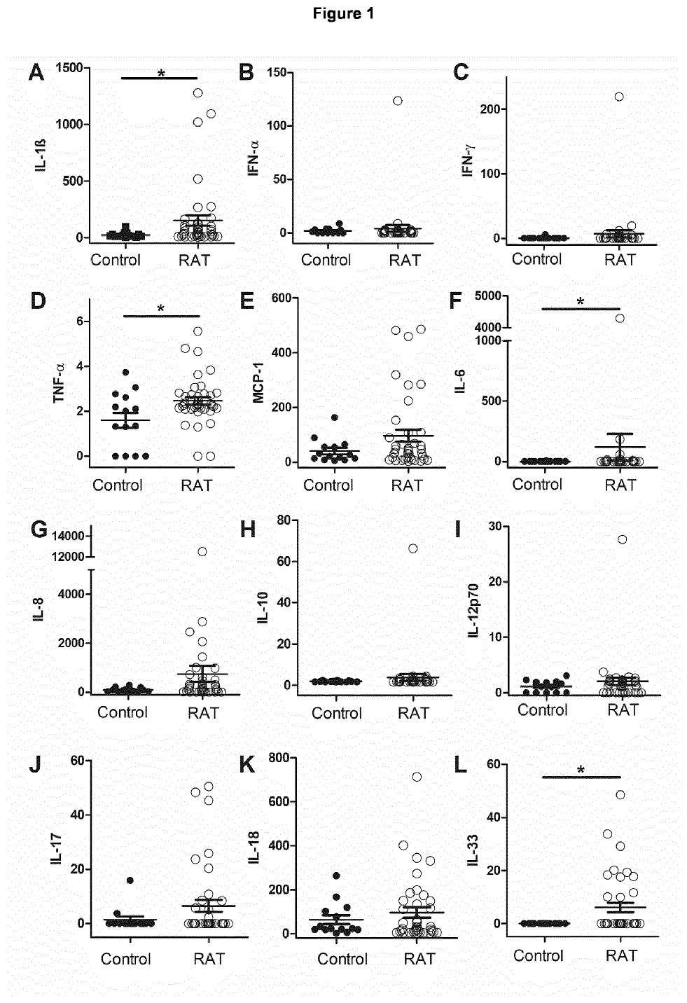

No differences between the healthy controls and the patients suffering from RAT could be found analyzing the salivary levels of IL8, IL-1β, IL-12p70, IL17A, IL-18, IFN-α, IFN-γ and MCP-1. Although salivary levels of IL-6, IL-33 and TNF-α were significantly increased in patients with recurrent tonsillitis compared to healthy controls, these parameters had no sufficient potential to differentiate between the diagnosis groups because of a high amount of false negative tests (FIG. 1 B-L). ROC analysis for IL-6, IL-33 and TNF-a revealed A-values <0.7 and confirmed their ineligibility as a potential biomarker (data not shown). Merely significantly increased levels of IL-1ß had the potential as an appropriate biomarker for recurrent tonsillitis (153.7±48.5 pg/ml vs 23.3±6.6 pg/ml, p=0.021) (FIG. 1 A). A cut-off value of 30 pg/ml was determined by ROC analysis to identify patients with recurrent tonsillitis with a sensitivity of 0.69 and a specificity of 0.88.

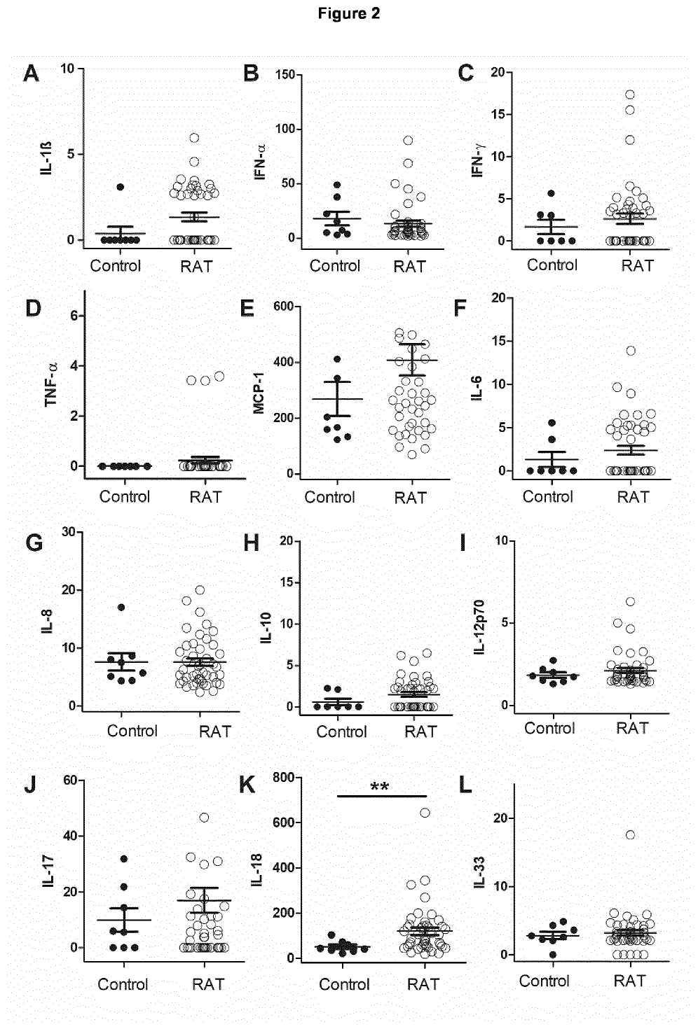

In serum significantly increased levels of IL-18 could be observed in patients with RAT compared to the controls (120.2±16.5 vs 50.6±9.3 pg/ml, p=0.007) (FIG. 2 K). IL-18 showed a sensitivity of 0.94 and a specificity of 0.63 to differentiate between healthy controls and patients suffering from RAT (cut-off: 44 pg/ml). Levels of IFN-α, IFN-γ, TNF-α, IL-6, IL8, IL-10, IL-12p70, IL17A, IL-33 and MCP-1 showed no significant differences between the healthy and the RAT group (p≥0.05) (FIG. 2 A-J, L).

### Example 2: S100AB/A9 Expression

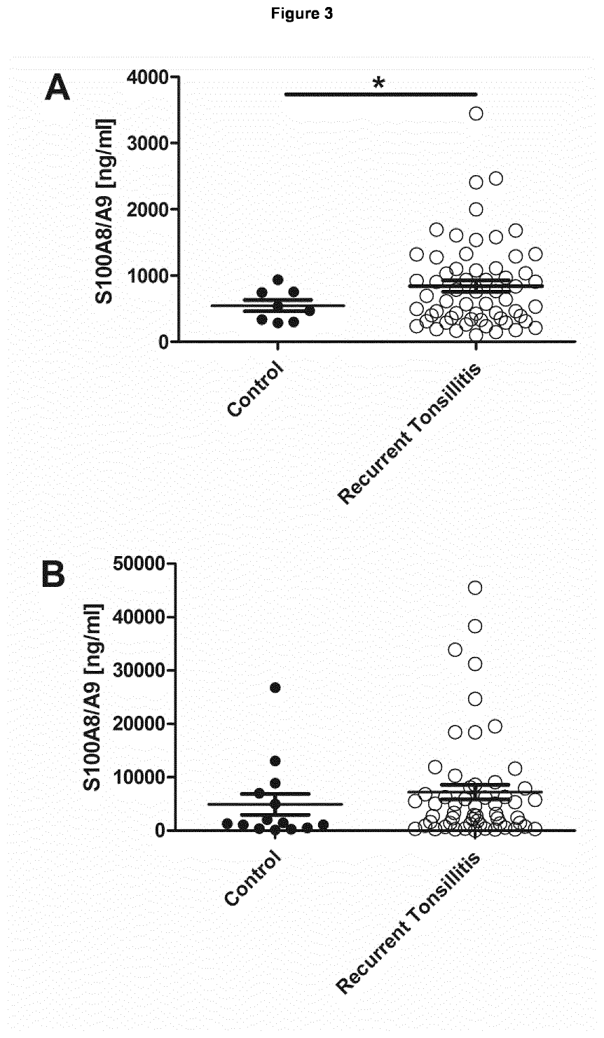

Significantly increased levels of S100A8/A9 could be observed in the serum (996.5±102.2 ng/ml vs 546.4±85.9 ng/ml, p=0.042) (FIG. 3 A) but not in the saliva of patients with RAT compared to the controls (8666.3±1636.1 ng/ml vs 4936.7±1975.8 ng/ml, p=0.072) (FIG. 3 B). ROC analysis revealed a cut-off value of 757 ng/ml of S100A8/A9 in serum to distinguish RAT and healthy controls with a sensitivity of 60% and a specificity of 88%.

### Example 3: Development of the RAT Score

With the purpose to establish an objective marker for a reliable identification of patients with recurrent tonsillitis a high sensitivity and specificity was required. Hence, cytokines and soluble proteins which have proven their potential to discriminate between RAT and controls by significant increased levels in saliva or serum and corresponding ROC analysis were combined to the RAT-score. For each parameter above the cut-off value of IL-1ß in saliva (30 pg/ml], IL-18 (44 g/ml), and S100A8/A9 (757 ng/ml) in serum one point is added to the RAT-score (Table 1). ROC analysis revealed a cut-off value of 1.5 for the existence of RAT with a sensitivity of 95% and a specificity of 88% (FIG. 4).

TABLE 1
RAT-score. One point for each elevated parameter
above the cut-off is added to determine the RAT-score.
Values ≥2 indicate the diagnosis of recurrent tonsillitis.

| | | cut-off | sensitivity | specificity | points |
|---|---|---|---|---|---|
| S100A8/A9 | Serum | 757 ng/ml | 0.60 | 0.88 | 1 |
| IL-1β | Saliva | 30 pg/ml | 0.69 | 0.88 | 1 |
| IL-18 | Serum | 44 pg/ml | 0.94 | 0.63 | 1 |
| RAT-score | | 1.5 points | 0.95 | 0.88 | 0-3 |

Using the data of Table 1, a person skilled in the art can conclude that an increased amount of at least two biomarkers of IL-1β, IL-18 and S100A8/S100A9 is indicative for a diagnosis of RAT, i.e. also for the necessity of tonsillectomy or tonsillotomy. Further, an increased amount of at least two biomarkers of IL-1β, IL-18 and S100A8/S100A9 could be indicative for an elevated high or a predisposition for developing RAT.

### REFERENCES

Alvarez K, Vasquez G. Damage-associated molecular patterns and their role as initiators of inflammatory and auto-immune signals in systemic lupus erythematosus. Int Rev Immunol 2017:1-12.
Bird J H, Biggs T C, King E V. Controversies in the management of acute tonsillitis: An evidence-based review. Clin Otolaryngol 2014; 39(6):368-74.
Brandtzaeg P, Halstensen T S. Immunology and immunopathology of tonsils. Adv Otorhinolaryngol 1992; 47:64-75.
Burton M J, Glasziou P P, Chong L Y, Venekamp R P. Tonsillectomy or adenotonsillectomy versus non-surgical treatment for chronic/recurrent acute tonsillitis. Cochrane Database Syst Rev 2014; (11):CD001802. doi(11):CD001802.
Douglas C M, Lang K, Whitmer W M, Wilson J A, Mackenzie K. The effect of tonsillectomy on the morbidity from recurrent tonsillitis. Clin Otolaryngol 2017.
Ebell M H, Smith M A, Barry H C, Ives K, Carey M. The rational clinical examination. does this patient have strep throat? JAMA 2000; 284(22):2912-8.
Ehrchen J M, Sunderkotter C, Foell D, Vogl T, Roth J. The endogenous toll-like receptor 4 agonist S100A8/S100A9 (calprotectin) as innate amplifier of infection, autoimmunity, and cancer. J Leukoc Biol 2009; 86(3):557-66.
Foell D, Wittkowski H, Vogl T, Roth J. S100 proteins expressed in phagocytes: A novel group of damage-associated molecular pattern molecules. J Leukoc Biol 2007; 81(1):28-37.
Freer J, Ally T, Brugha R. Impact of cantor scores on determining antibiotic prescribing in children. Int J Health Care Qual Assur 2017; 30(4):319-26.
Frosch M, Strey A, Vogl T, Wulffraat N M, Kuis W, Sunderkotter C, et al. Myeloid-related proteins 8 and 14 are specifically secreted during interaction of phagocytes and activated endothelium and are useful markers for monitoring disease activity in pauciarticular-onset juvenile rheumatoid arthritis. Arthritis Rheum 2000; 43(3):628-37.
Gulliford M C, Dregan A, Moore M V, Ashworth M, Staa T, McCann G, et al. Continued high rates of antibiotic prescribing to adults with respiratory tract infection: Survey of 568 UK general practices. BMJ Open 2014; 4(10):e006245, 2014-006245.
Hellings P, Jorissen M, Ceuppens J L. The waldeyer's ring. Acta Otorhinolaryngol Belg 2000; 54(3):237-41.
Koskenkorva T, Koivunen P, Penna T, Teppo H, Alho O P. Factors affecting quality-of-life impact of adult tonsillectomy. J Laryngol Otol 2009; 123(9):1010-4.
Leukert N, Vogl T, Strupat K, Reichelt R, Sorg C, Roth J. Calcium-dependent tetramer formation of S100A8 and S100A9 is essential for biological activity. J Mol Biol 2006; 359(4):961-72.
Morad A, Sathe N A, Francis D O, McPheeters M L, Chinnadurai S. Tonsillectomy versus watchful waiting for recurrent throat infection: A systematic review. Pediatrics 2017; 139(2):10.1542/peds.2016, 3490. Epub 2017 Jan. 17.
Mui S, Rasgon B M, Hilsinger R L,Jr. Efficacy of tonsillectomy for recurrent throat infection in adults. Laryngoscope 1998; 108(9):1325-8.
Nave H, Gebert A, Pabst R. Morphology and immunology of the human palatine tonsil. Anat Embryol (Ber) 2001; 204(5):367-73.
Paradise J L, Bluestone C D, Bachman R Z, Colbom D K, Bemard B S, Taylor F H, et al. Efficacy of tonsillectomy for recurrent throat infection in severely affected children. results of parallel randomized and nonrandomized clinical trials. N Engl J Med 1984; 310(11):674-83.
Paulussen C, Claes J, Claes G, Jorissen M. Adenoids and tonsils, indications for surgery and immunological consequences of surgery. Acta Otorhinolaryngol Belg 2000; 54(3):403-8.
Perry M, Whyte A. Immunology of the tonsils. Immunol Today 1998; 19(9):414-21.
Ross K F, Herzberg M C. Calprotectin expression by gingival epithelial cells. Infect Immun 2001; 69(5):3248-54.
Schlegel Gomez R, Langer P, Pelka M, von den Driesch P, Johannessen A C, Simon M,Jr. Variational expression of functionally different macrophage markers (27E10, 25F9, RM3/1) in normal gingiva and inflammatory periodontal disease. J Clin Periodontol 1995; 22(5):341-6.
Senska G, Atay H, Putter C, Dost P. Long-term results from tonsillectomy in adults. Dtsch Arztebl Int 2015; 112(50):849-55.
Slaats J, Ten Oever J, van de Veerdonk F L, Netea M G. IL-1beta/IL-6/CRP and IL-18/ferritin: Distinct inflammatory programs in infections. PLoS Pathog 2016; 12(12):e1005973.
So A K, Martinon F. Inflammation in gout: Mechanisms and therapeutic targets. Nat Rev Rheumatol 2017.
Sorenson B S, Khammanivong A, Guenther B D, Ross K F, Herzberg M C. IL-1 receptor regulates S100A8/A9-dependent keratinocyte resistance to bacterial invasion. Mucosal Immunol 2012; 5(1):66-75.
Spiekermann C, Russo A, Stenner M, Rudack C, Roth J, Vogl T. Increased levels of S100A8/A9 in patients with peritonsillar abscess: A new promising diagnostic marker to differentiate between peritonsillar abscess and peritonsillitis. Disease Markers, 2017, doi: 10.1155/2017/9126560.
Stelter K. Tonsillitis and sore throat in children. GMS Curr Top Otorhinolaryngol Head Neck Strupat K, Rogniaux H, Van Dorsselaer A, Roth J, Vogl T. Calcium-induced noncovalently linked tetramers of MRP8 and MRP14 are confirmed by electrospray ionization-mass analysis. J Am Soc Mass Spectrom 2000; 11(9):780-8.
Surg 2014; 13:Doc07.
Sudan L, Jr. Tonsils and lympho-epithelial structures in the pharynx as immuno-barriers. Acta Otolaryngol 1987; 103(5-6):369-72.
Tyden H, Lood C, Gullstrand B, Jonsen A, Nived O, Sturfelt G, et al. Increased serum levels of S100A8/A9 and S100A12 are associated with cardiovascular disease in patients with inactive systemic lupus erythematosus. Rheumatology (Oxford) 2013; 52(11):2048-55.
van den Bosch M H, Blom A B, Schelbergen R F, Koenders M I, van de Loa F A, van den Berg W B, et al. Alarmin S100A9 induces proinflammatory and catabolic effects predominantly in the M1 macrophages of human osteoarthritic synovium. J Rheumatol 2016; 43(10):1874-84.
Williams A, Lee P, Kerr A. Scottish intercollegiate guidelines network (SIGN) guidelines on tonsillectomy: A three cycle audit of clinical record keeping and adherence to national guidelines. J Laryngol Otol 2002; 116(6):453-4.
Windfuhr J P, Toepfner N, Steffen G, Waldfahrer F, Bemer R. Clinical practice guideline: Tonsillitis II. surgical management. Eur Arch Otorhinolaryngol 2016; 273(4):989-1009.

1. A method of stratifying a subject having no symptoms of acute tonsillitis for the necessity of tonsillectomy or tonsillotomy, comprising:
(i) determining the amount of the biomarkers IL-1β, IL-18 and S100A8/S100A9 in a sample obtained from said subject, and
(ii) stratifying said subject for the necessity of tonsillectomy or tonsillotomy by comparing the amount of said biomarkers with a threshold value for each biomarker.

2. The method according to claim 1, wherein an increased amount of at least two of said biomarkers relative to the respective threshold value is indicative for the necessity for tonsillectomy or tonsillotomy in said subject.

3. The method according to claim 1, wherein no increased amount of at least two of said biomarkers relative to the respective threshold value indicates that there is no necessity for tonsillectomy or tonsillotomy in said subject.

4. A method of diagnosing recurrent tonsillitis in a subject having no symptoms of acute tonsillitis, comprising:
(i) determining the amount of the biomarkers IL-1β, IL-18 and S100A8/S100A9 in a sample obtained from said subject, and
(ii) diagnosing recurrent tonsillitis in said subject by comparing the amount of said biomarkers with a threshold value for each biomarker.

5. The method according to claim 4, wherein an increased amount of at least two of said biomarkers relative to the respective threshold value is indicative for the presence of recurrent tonsillitis in said subject.

6. The method according to claim 4, wherein no increased amount of at least two of said biomarkers relative to the respective threshold value indicates that there is no presence of recurrent tonsillitis in said subject.

7. A method of predicting the risk or the predisposition for recurrence of acute tonsillitis (RAT) in a subject having no symptoms of acute tonsillitis, comprising:
(i) determining the amount of the biomarkers IL-1β, IL-18 and S100A8/S100A9 in a sample obtained from said subject, and
(ii) predicting the risk or the predisposition for recurrence of acute tonsillitis by comparing the amount of said biomarkers with a threshold value for each biomarker.

8. The method according to claim 7, wherein an increased amount of at least two of said biomarkers relative to the respective threshold value is indicative for an elevated risk or a predisposition for recurrence of acute tonsillitis.

9. The method according to claim 7, wherein no increased amount of at least two of said biomarkers relative to the respective threshold value indicates no elevated risk or no predisposition for recurrence of acute tonsillitis.

10. The method according to any one of claims 1 to 9, wherein the subject having no symptoms of acute tonsillitis does not fulfil the criterion of having at least six episodes of diagnosed purulent acute tonsillitis in the previous year, wherein each episode is preferably characterized by administration of antibiotics.

11. The method according any one of claims 1 to 9, wherein the subject having no symptoms of acute tonsillitis fulfils the criterion of having at least six episodes of diagnosed purulent acute tonsillitis in the previous year, wherein each episode is preferably characterized by administration of antibiotics.

12. The method according to any one of claims 1 to 11, wherein said acute tonsillitis is a bacterial or viral tonsillitis.

13. The method according to any of claims 1 to 12, wherein the sample is a saliva sample, a serum sample, a tissue sample, a blood sample, a urine sample, a lymphatic fluid sample, a nasopharyngeal wash sample, a sputum sample, a mouth swab sample, a throat swab sample, a nasal swab sample, a bronchoalveolar lavage sample, or a bronchial secretion sample.

14. The method according to any one of claims 1 to 13, wherein the amount of S100A8/S100A9 and IL-18 is determined in a serum sample and wherein the amount of IL-1β is determined in a saliva sample.

15. The method according to any one of claims 1 to 14, wherein the amount of S100A8/S100A9, IL-1β and IL-18 is determined by using a multiplex bead-based assay, mass spectrometry, chromatography, turbidity assay, lateral flow technology and/or an immunoassay.

16. The method according to any one of claims 1 to 15, wherein the threshold value is a predetermined threshold value.

17. The method according to any one of claims 1 to 16, wherein the threshold value is calculated by comparing the amount of the at least two biomarkers in a group of patients diagnosed with recurrence of acute tonsillitis (RAT) and having no symptoms of acute tonsillitis with the amount of said biomarker in a healthy control group.

18. The method according to claim 17, wherein the patients diagnosed with recurrence of acute tonsillitis (RAT) fulfil the criterion of having at least six episodes of diagnosed purulent acute tonsillitis in the previous year, wherein each episode is preferably characterized by administration of antibiotics.

19. The method according to any one of claims 1 to 18, wherein the threshold value is calculated by ROC analysis, wherein the area under the curve (A-value) is at least 0.7, preferably at least 0.8, more preferably at least 0.9.

20. The method according to any of claims 1 to 19, wherein the symptoms of acute tonsillitis are sore throat, red and swollen tonsils and pain when swallowing.

21. The method according to any one of claims 1 to 20, wherein the subject is a mammal, preferably a human subject.

22. The method according to any one of claims 1 to 21, wherein said subject is an adult, a juvenile or a child.
